(19) United States
(12) Patent Application Publication
(10) Pub. No.: US 2005/0272963 A1
(43) Pub. Date: Dec. 8, 2005

(54) Method for the production of acetylene alcohols

(75) Inventors: Jochem Henkelmann, Mannheim (DE); Alois Kindler, Grunstadt (DE); Jan-Dirk Arndt, Mannheim (DE); Katrin Klass, Mannheim (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 10/523,925

(22) PCT Filed: Jul. 23, 2003
(86) PCT No.: PCT/EP03/08045

§ 371 Date: Feb. 7, 2005

(30) Foreign Application Priority Data
Aug. 8, 2002 (DE) 102 36 578.4

(51) Int. Cl. C07C 33/04; C07F 1/02

(52) U.S. Cl. 568/874; 260/665 R

(57) ABSTRACT

The present invention relates to a process for preparing acetylene alcohols of the general formula I 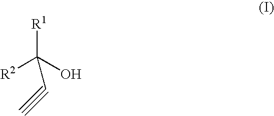 where
R¹ and R² may be the same or different and are each independently hydrogen, a saturated or a mono- or polyunsaturated C₁-C₃₀-alkyl, aryl, cycloalkylalkyl or cycloalkyl radical, each of which may optionally be substituted, or a group of the general formula (II) 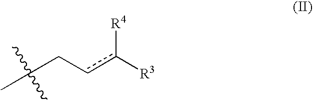 where
R³ and R⁴ may be the same or different and are each independently hydrogen or a saturated or a mono- or polyunsaturated C₁-C₃₀-alkyl, aryl, cycloalky alkyl or cycloalkyl radical, each of which may optionally be substituted, and the dashed line may represent an additional double bond,
by monoethynylating a ketone of the general formula R¹—CO—R² by
(a) reacting lithium with a C₁-C₁₀-alkyl halide (b) feeding in acetylene gas (c) adding the ketone.

The present invention relates to a process for preparing acetylene alcohols by monoethynylating a ketone by reacting an alkyl halide with lithium.

The state of the art is the continuously operated ethynylation of ketones with acetylene in liquid ammonia with catalytic amounts of base (usually KOH or potassium methoxide in a polar, protic solvent; 10-40° C.; 20 bar), as described, for example, in DE 12 32 573.

In a further process for the 1,2-ethynylation of α,β-unsaturated ketones, monolithium acetylide complex is reacted with the appropriate carbonyl compound in an inert organic solvent (CH 642 936). The active lithium acetylide-ammonia complex is prepared by evaporating ammonia out of the lithium acetylide-ammonia solution at from −30 to −20° C. and replacing it with an organic solvent. An alternative process detailed is the reaction of lithium amide with acetylene in an inert organic solvent.

U.S. Pat. No. 2,472,310 describes a method for ethynylating rapidly aldolizing ketones, for example β-ionone, under basic conditions. The lithium acetylide-ammonia complex required for this purpose is prepared by passing acetylene into liquid ammonia at −40° C. and adding lithium at the same time (O. A. Shavrygina, D. V. Nazarova, S. M. Makin, Zh. Org. Khim. 1966, 2, 1566-1568).

A disadvantage of the processes mentioned is the low selectivity of lithium acetylide formation, since the lithium acetylide may be present as the mono- or dilithium acetylide or as a mixture of the two components. A further disadvantage is the low temperature required in order to maintain the ammonia in liquid form and the solvent exchange after the lithium acetylide formation.

U.S. Pat. No. 2,425,201 discloses a process for preparing α,β-unsaturated ketones using calcium acetylides. The ethynylation is carried out at temperatures of from −70 to −40° C.

E 10 81 883 describes a process for preparing ethynylionol by reacting sodium acetylide with β-ionone in an organic solvent. To increase the acetylene concentration in the reaction mixture, acetylene is used under pressure. Compared to the atmospheric pressure method, ethynylionol is obtained in an improved yield.

In a further process (DE 17 68 877), the preparation is described of acetylene alcohols by reacting sodium ethoxide with acetylene and an appropriate ketone in an organic solvent under pressure, at approx. 14 bar. However, working under pressure is to be regarded as a distinct disadvantage in this process in view of safety when working with acetylene and the associated costs.

Instead of lithium in liquid ammonia, it is also possible to use sodium, although the ketone likewise has to be added in another solvent after the sodium acetylide formation, so that the ammonia evaporates off slowly (P. Karrer, J. Benz, Helv. Chim. Acta 1948, 31, 390-295).

In another process, the synthesis of lithium acetylide is based on the reaction of lithium with naphthalene and acetylene, initially forming a naphthalene radical anion by electron transfer which then acts as a base and forms the lithium acetylide with acetylene. The reaction with β-ionone then gives the desired ethynylionol in a 90% yield (K. Suga, S. Watanabe, T. Suzuki, Can. J. Chem. 1968, 46, 3041-3045). The use of semistoichiometric amounts of naphthalene based on β-ionone is disadvantageous here.

A catalytic process for synthesizing alkyllithium compounds is also known. In the presence of 4,4′-di-tert-butylphenyl as a catalyst, lithium forms a radical anion by simple electron transfer which then forms the corresponding alkyllithium species by reaction with alkyl halides (P. K. Freeman, L. L. Hutchinson, Tetrahedron Letters, 1976, 22, 1849-1852; P. K. Freeman, L. L. Hutchinson, J. Org. Chem. 1983, 48, 4705-4713). Naphthalene may also be used as a catalyst in some cases. Preference is given to using the alkyllithium compound obtained in the reaction for alkylating various electrophiles (M. Yus, D. Ramon, J. Chem.

Soc., Chem. Comm. 1991, 398-400; T. R. van den Ancker, M. J. Hdgson, J. Chem. Soc., Perkin Trans. 1, 1999, 2869-2870).

It is an object of the present invention to develop an economical process for preparing acetylene alcohols which does not have the disadvantages described in the prior art.

We have found that this object is achieved according to the invention, surprisingly, by a one-pot process for preparing acetylene alcohols of the general formula I 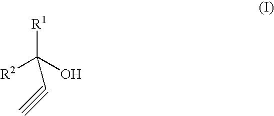
where
R¹ and R² may be the same or different and are each independently hydrogen, a saturated or a mono- or polyunsaturated C₁-C₃₀-alkyl, aryl, cycloalkylalkyl or cycloalkyl radical, each of which may optionally be substituted, or a group of the general formula (II) 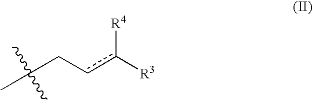
   where
R³ and R⁴ may be the same or different and are each independently hydrogen or a saturated or a mono- or polyunsaturated C₁-C₃₀-alkyl, aryl, cycloalkylalkyl or cycloalkyl radical, each of which may optionally be substituted, and the dashed line may represent an additional double bond,
by monoethynylating a ketone of the general formula R¹—CO—R² by
(a) reacting lithium with a C₁-C₁₀-alkyl halide
(b) feeding in acetylene gas
(c) adding the ketone.

Preference is given to reacting lithium with alkyl halide in the presence of catalytic amounts of naphthalene or 4,4′-di-tert-butylbiphenyl. The solvent used for this purpose may be tetrahydrofuran.

A C₁-C₄-alkyl radical is a methyl, ethyl, propyl, i-propyl, butyl or t-butyl radical.

A mono- or polyunsaturated straight-chain or branched C₁-C₃₀-alkyl radical is, for example, unless otherwise stated, a methyl, ethyl, propyl, isopropyl, n-butyl, i-butyl, t-butyl, pentyl, hexyl, heptenyl, octyl, nonyl, decyl, 1-propenyl, 2-propenyl, 2-methyl-2-propenyl, 1-pentenyl, 1-methyl-2-pentenyl, isopropenyl, 1-butenyl, hexenyl, heptenyl, octenyl, nonenyl or decenyl radical, or the radicals corresponding to the compounds listed hereinbelow.

Cycloalkyl is a 3-7-membered saturated or a mono- or polyunsaturated 3-7-membered ring in which a CH₂ group may be replaced by O or NH, such as, inter alia, the cyclopropyl, cyclobutyl, cyclopentyl, cyclohexyl or cycloheptyl ring, preferably the cyclopentyl or cyclohexyl ring.

An aryl radical is preferably a benzyl, phenyl or naphthyl radical.

In addition to C₁-C₄-alkyl, further substituents may be methyl, ethyl, propyl, i-propyl, butyl, t-butyl, fluorine, chlorine, bromine, iodine, nitro or amino radicals.

The following ketones, for example, may be used for ethynylation:

acetone, methyl vinyl ketone, β-ionone, tetrahydrogeranylacetone, 6-methylheptanone, hexahydrofarnesylacetone, diethyl ketone, methyl ethyl ketone, cyclohexanone, methyl t-butyl ketone, pseudoionone, methylhexenone and H-geranylacetone, preferably acetone, methyl vinyl ketone or β-ionone.

In the first step of the process according to the invention, alkyllithium is generated in situ by reacting lithium with an alkyl halide, for example 1-chlorobutane, in the presence of catalytic amounts (12.5 mol %) of 4,4′-di-tert-butylbiphenyl at temperatures of from −20 to −10° C., preferably at −15° C. After the excess lithium is removed from the reaction mixture by filtration, acetylene gas is introduced to prepare lithium acetylide.

The last step of the one-pot reaction is the addition of the ketone at 0 to 10° C., preferably at 0° C. Surprisingly, no disproportionation of the lithium acetylide into dilithium acetylide and acetylene takes place.

The solvent used in this process may be tetrahydrofuran.

In the reaction according to the invention, the exclusive formation of the monolithium acetylide species was observed, while the reaction of commercial alkyllithium, for example butyllithium, with acetylene above −25° C. resulted in the observation of a disproportionation to give acetylene and insoluble dilithium acetylide. At 0° C., dilithium acetylide in tetrahydrofuran is in equilibrium with the monoacetylide species, and the addition of an electrophile results in the shifting of the equilibrium to obtain the corresponding ethynylated species.

The process according to the invention allows acetylene alcohols to be prepared without any problems in good to very good yields starting, for example, from the ketones acetone, β-ionone or methyl vinyl ketone. The ethynylation products of β-ionone and methyl vinyl ketone are precursors of vitamin A and the astaxanthine synthesis.

In addition to the reaction of the ketones according to the invention, it is also possible to react trimethylsilyl chloride to synthesize trimethylsilylacetylene. Trimethylsilylacetylene are used for synthesizing enediynes which are active as antitumor reagents.

The following examples are intended to illustrate the invention, without restricting it thereto:

### EXAMPLES

The reaction is carried out in two 250 ml HWS vessels under argon. First, 2.4 g (0.34 mol) of lithium wire are cut into small pieces and suspended together with 5.4 g (20 mmol) of the catalyst in 200 ml of tetrahydrofuran at −15° C. Once an intensive blue coloration of the reaction mixture has developed, 14.8 g (0.16 mol) of 1-chlorobutane in 20 ml of tetrahydrofuran are added via a dropping funnel within two hours and then stirred for a further two hours. The lithium is removed by transferring the supernatant solution into a second 250 ml HWS vessel, into which a 4 l/h stream of acetylene is introduced at −15° C. (1.5 h). After the lithium acetylide formation, 0.18 mol of the corresponding ketone in 20 ml of tetrahydrofuran is added dropwise via a dropping funnel at 0° C. within two hours. After heating to room temperature, hydrolysis is effected by adding water and the phases are separated.

| | | | | Conversion | Selectivity | | |
|---|---|---|---|---|---|---|---|
| Ex. | Cat. | Ketone | Product | [%] | [%] | Yield [%] | Comment |
| 1 | Biph | Acetone | MBY | 100 | 81.0 | 81.0 | — |
| 2 | Biph | MVK | VBY | 98.1 | 59.1 | 57.9 | Dropwise addition of |
| | | | | | | | MVK at −10° C., |
| | | | | | | | owing to |
| | | | | | | | polymerization |
| 3 | Biph | β-Ionone | Ethynyl- | 99.4 | 78.9 | 78.4 | Stirring for 16 h after |
| | | | ionol | | | | β-ionone addition |
| 4 | Biph | TMS-Cl | TMS- | 99.2 | 92.1 | 91.4 | — |
| | | | Acetylene | | | | |
| 5 | Naph | Acetone | MBY | 98.9 | 57.1 | 56.5 | NP: Alkylation of naph |
| 6 | Biph | 1,2-Epoxy- | — | — | — | — | No product |
| | | butane | | | | | |
| 7 | Biph | Butyl | — | — | — | — | No product |
| | | chloro- | | | | | |
| | | formate | | | | | |
| 8 | Biph | Methyl | — | — | — | — | No product |
| | | chloro- | | | | | |
| | | formate | | | | | |

MBY: Methylbutynol
MVK: Methyl vinyl ketone
VBY: Vinylbutynol
Biph: 4,4′-Di-tert-butylbiphenyl
Naph: Naphthalene

1. A process for preparing an acetylene alcohol of the general formula I: 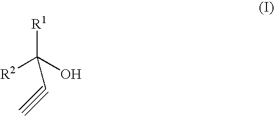
wherein
R¹ and R² may be the same or different and are each independently a saturated or a mono- or polyunsaturated C₁-C₃₀-alkyl, aryl, cycloalkylalkyl or cycloalkyl radical, each of which may optionally be substituted, or a group of the general formula (II): 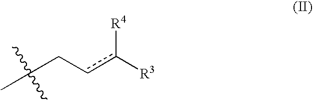
wherein
R³ and R⁴ may be the same or different, and are each independently hydrogen or a saturated or a mono- or polyunsaturated C₁-C₃₀-alkyl, aryl, cycloalkylalkyl or cycloalkyl radical, each of which may optionally be substituted, and the dashed line may represent an additional double bond,
said process comprising monoethynylating a ketone of the general formula
R¹—CO—R² by
(a) reacting lithium with a C₁-C₁₀-alkyl halide
(b) feeding in acetylene gas
(c) adding the ketone.

2. The process as claimed in claim 1, wherein the reaction of lithium with the C₁-C₁₀-alkyl halide is carried out in the presence of catalytic amounts of naphthalene or 4,4′-di-tert-butylbiphenyl.

3. The process as claimed in claim 1, wherein the ketone is selected from the group consisting of acetone, methyl vinyl ketone, β-ionone, tetrahydrogeranylacetone, 6-methylheptanone, hexahydrofarnesylacetone, diethyl ketone, methyl ethyl ketone, cyclohexanone, methyl t-butyl ketone, pseudoionone, methylhexenone and H-geranylacetone.

4. The process as claimed in claim 2, wherein the ketone is selected from the group consisting of acetone, methyl vinyl ketone, β-ionone, tetrahydrogeranylacetone, 6-methylheptanone, hexahydrofarnesylacetone, diethyl ketone, methyl ethyl ketone, cyclohexanone, methyl t-butyl ketone, pseudoionone, methylhexenone and H-geranylacetone.
